(19) United States
(12) Patent Application Publication
(10) Pub. No.: US 2020/0111576 A1
(43) Pub. Date: Apr. 9, 2020

(54) PRODUCING A MULTIDIMENSIONAL SPACE DATA STRUCTURE TO PERFORM SURVIVAL ANALYSIS

(71) Applicant: Babylon Partners Limited, London (GB)

(72) Inventors: Christopher Robert HART, London (GB); Jonathan George RICHENS, London (GB)

(21) Appl. No.: 16/276,330

(22) Filed: Feb. 14, 2019

Related U.S. Application Data
(62) Division of application No. 16/152,093, filed on Oct. 4, 2018.

(51) Int. Cl. G16H 50/30; G06N 3/04; G06N 3/08

(52) U.S. Cl. CPC G16H 50/30; G06N 3/08; G06N 3/0454; G06N 3/0472

(57) ABSTRACT

Computer implemented methods and systems of using a trained probabilistic graphical model to predict whether a user will develop a health condition are provided. The method includes representing functional dependencies of first and second statistical models as neural networks. The method also includes receiving training data comprising time to event data with corresponding intervention data and observable variables. The method also includes training said neural networks using said training data.

### CROSS-REFERENCE TO RELATED APPLICATION

The present application claims the benefit of priority under 35 U.S.C. § 120 as a divisional from U.S. patent application Ser. No. 16/152,093, entitled “PRODUCING A MULTIDIMENSIONAL SPACE DATA STRUCTURE TO PERFORM SURVIVAL ANALYSIS,” filed on Oct. 4, 2018, the disclosure of which is hereby incorporated by reference in its entirety for all purposes.

### FIELD

Embodiments described herein relate to processing a multidimensional observable variable space data structure to produce a new multidimensional space data structure that is sampled to determine when an event will occur in order to perform survival analysis.

### BACKGROUND

Survival analysis relates to methods where the outcome of variable is the time until the occurrence of the event of interest. It can be used in healthcare applications to track predicted time to developing a disease or death, where leaving the study would constitute a censoring event.

### BRIEF DESCRIPTION OF FIGURES

FIG. 1 is a schematic of a system in accordance with an embodiment;

FIG. 2 is a flow chart depicting a method in accordance with an embodiment;

FIG. 3 is a flow chart depicting a method in accordance with a further embodiment;

FIG. 4 is a probabilistic graphical model for survival analysis used in accordance with an embodiment;

FIG. 5 is a diagram showing a neural network structure in accordance with an embodiment with reference to the latent space;

FIG. 6 is a diagram showing a neural network structure with reference to the proxy variables through to the output; and

FIG. 7 is a schematic of a system in accordance with an embodiment,

### DETAILED DESCRIPTION OF FIGURES

In an embodiment, a computer implemented method is provided of using a trained probabilistic graphical model to predict whether a user will develop a health condition, the method comprising:

a. retrieving data concerning the user,
b. inputting the retrieved data into a trained model, the trained model being a probabilistic graphical model comprising an observable variable space, a latent variable space and an outcome relating to said condition, wherein the observable multidimensional variable space is dependent on the multidimensional latent variable space and the likelihood of a user developing a condition is dependent on the multidimensional latent variable space, wherein the trained model has been trained using observational training data wherein said observational training data comprises observations regarding individuals developing said condition;
c. using said trained model to output if and when the user is likely to develop the condition.

The disclosed system and methods provides an improvement to computer functionality by allowing computer performance of a function not previously performed by a computer. Specifically, the disclosed system provides for constructing a multidimensional latent variable space from an observable multidimensional space, the system then allowing for the processing a data structure in the form of a multidimensional observable variable space to produce a new data structure in the form of a multidimensional space. A first statistical model is used to define the link between the multidimensional variable space and the multidimensional latent space. A second statistical model is used to define the link between the outcome (time to event) the multidimensional latent space and an intervention. The method then allows for sampling from this latent variable space to determine when an event will occur and how intervening on a specific risk factor will change that risk. In an embodiment, a neural network architecture is used to represent the functional dependencies of the first and second statistical models.

The above method has implications in the medical field and it will allow survival analysis to be performed with results tailored to an individual. For example, it is possible to determine the likelihood of a patient suffering from heart disease dependent on observable parameters such as their age, location, socio economic group. The disclosed system addresses this problem by the structure of the model and the learned relationship between the multidimensional latent variable space and the observable variable multidimensional space.

The disclosed system also addresses a technical problem tied to computer technology, namely the technical problem of the efficient use of data of processor capacity since the system can allow a new data structure to be produced that allows more efficient processing of the data and reduction in required memory. The modelling of the data in the way presented in the embodiments, allows training data to be used where the condition was not developed during the collection of the training data. This is achieved via the modelling using an event flag to indicate whether the condition was observed or not and the time of the observation of the event if the event was observed and the time at which observation was stopped if the event was not observed. Thus, not only data where the event was observed is used for training, but also data where the event was not observed.

In a further embodiment, the model further comprises an intervention variable used to model intervention and wherein the likelihood of a user developing a condition is dependent on the latent variable space and the intervention variable. The use of the intervention variable allows the model to model the effect of a treatment and thus the user can obtain individually-tailored predictions on the likely time that they will develop heart disease dependent on certain treatments or interventions, for example, if they take statins, exercise daily, reduce their alcohol etc.

This intervention can be modelled as a time to event variable.

It is assumed that there is a latent multidimensional space that is defined by latent variables from which the time to event variable can be derived both with and without the effect of an intervention. These latent variables are not observable, but proxy variables can be observed that are affected by the latent variables and these proxy variables can be observed. By observing these proxy variables, it is possible to obtain information about the latent space,

In an embodiment, the probability of the time to event variable over the intervention variable and the latent variable space is an antisymmetric distribution. In a further embodiment, the distribution is a Weibull distribution. This can be used where the time to event variable is continuous. In further embodiments, the time to event variable is represented as one of a plurality of labels, for example, 30-39, 40-49 etc. The system can model time in discrete units, so rather than being able to get a risk prediction for 1.5672947 years into the future, the system would be able to predict an outcome e.g. 1-2 years, 2-3 years, and so on, up to some pre-defined maximum (e.g. 30+ years). Here, the probability distribution can be represented as a categorical distribution.

In further embodiments, a neural network is used to model the relationship between the time to event variable, the latent variable space and the intervention variable.

The latent variable space may comprise both discrete and continuous variables. Further,

The multivariable latent space may be drawn from a multivariate Normal distribution.

In an embodiment, the multivariable latent space comprises discrete variables and the observable variables are linked to the discrete variables of the multivariable latent space via a Bernoulli probability distribution. In a further embodiment, the multivariable latent space comprises continuous variables and the observable variables are linked to the continuous variables of the multivariable latent space via a normal probability distribution. The model may comprise a neural network to model the relationship between the multivariable latent space and the observable variables.

As mentioned above, the proxy variables allow information to be determined about the latent space, the proxy variables may be, for example, age of the user, prior medical history, e.g. whether they have suffered from certain conditions, where they live, socio economic group, family history etc. The importance of these variables will depend on the question being asked for example questions about heart disease and diabetes will have different important proxy variables. The proxy variables will have “default distributions” of what they could be, which are then improved by the data that is available when reconstructing the multivariate latent space. From those distributions a “default value” can be used in the absence of data. These default values will then be set to values retrieved for the user. Not all values will need to be changed from their default value to values specific to the user. The method may be adapted to determine if the data retrieved concerning the user is sufficient to determine if the user will develop the condition and requesting further information if the data is not sufficient. In one embodiment, the method determines a confidence estimate on the output and to request further information if the confidence estimate is below a threshold.

In an embodiment, data concerning the user will comprise at least the user's age. In further embodiments, data concerning the user is received from a fitness tracker or the like.

The above has discussed determining the time to event for a user and also that this time to event can be determined both in the presence of and in the absence of a treatment or intervention. From this, it is possible to determine the effect of a treatment on a user. However, it is possible to estimate the average treatment effect for a treatment, wherein the treatment is represented as the intervention and the change in a time to event using the treatment is calculated for a plurality of users and the average is calculated.

In a further embodiment, a method of training a model is provided, the model being a probabilistic graphical model used to predict whether a user will develop a health condition, the model comprising an observable variable space, a latent variable space, an intervention variable space and a time to event variable, said time to event variable indicating when user is likely to develop a condition, wherein the observable variable space is dependent on the latent variable space and the time to event variable is dependent on the latent variable space and intervention variable space, the model comprising a first statistical model comprising probability distributions linking the observable variable space to the latent variable space and a second statistical model comprising probability distributions linking the time to event variable to the latent variable space and intervention variable space, the method comprising representing the functional dependencies of the first and second statistical models as neural networks; receiving training data comprising time to event data with corresponding intervention data and observable variables; and training said neural networks using said training data.

In a yet further embodiment, a computer implemented method is provided to predict whether a user will develop a health condition, the method comprising:

a. training a model, using observational training data wherein said observational training data comprises observations regarding individuals developing said condition, the model being a probabilistic graphical model comprising an observable variable space, a latent variable space and an outcome relating to said condition, wherein the observable multidimensional variable space is dependent on the multidimensional latent variable space and the likelihood of a user developing a condition is dependent on the multidimensional latent variable space;
b. retrieving data concerning the user,
c. inputting the retrieved data into said model; and
d. using said model to output if and when the user is likely to develop the condition.

In a yet further embodiment, a computer implemented method is provided of using a probabilistic graphical model to predict whether a user will develop a health condition, the method comprising:

a. retrieving data concerning the user,
b. inputting the retrieved data into a model, the model being a probabilistic graphical model comprising an observable variable space, a latent variable space and an outcome relating to said condition, wherein the observable multidimensional variable space is dependent on the multidimensional latent variable space and the likelihood of a user developing a condition is dependent on the multidimensional latent variable space; and
c. using said trained model to output if and when the user is likely to develop the condition.

In a further embodiment, a system for predicting if and when a user will develop a health condition, the system comprising an interface, a processor and memory:

a. the interface being adapted to receive a query from a user concerning their time to develop a condition and receive data concerning the user,
b. the processor being adapted to input the retrieved data into a trained model provided in the memory, the trained model being a probabilistic graphical model comprising an observable variable space, a latent variable space and an outcome relating to said condition, wherein the observable variable space is dependent on the latent variable space and the likelihood of a user developing a condition is dependent on the latent variable space, wherein the trained model has been trained using observational training data wherein said observational training data comprises observations regarding individuals developing said condition,
c. the interface being adapted to output from said trained model if and when the user is likely to develop the condition.

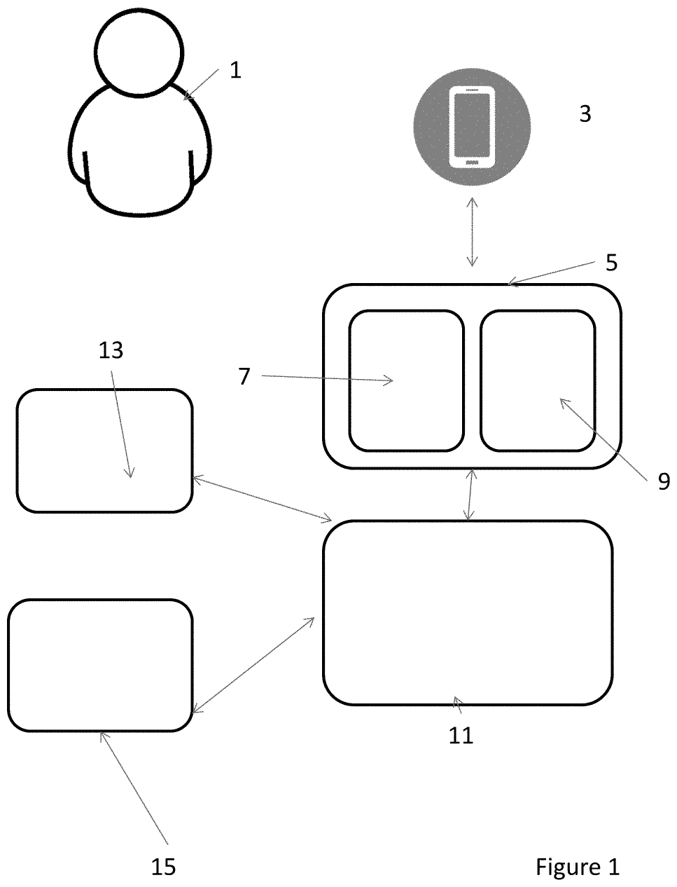

FIG. 1 is a schematic of a diagnostic system. In one embodiment, a user 1 communicates with the system via a mobile phone 3. However, any device could be used, which is capable of communicating information over a computer network, for example, a laptop, tablet computer, information point, fixed computer etc.

The mobile phone 3 will communicate with interface 5. Interface 5 has 2 primary functions, the first function 7 is to take the words uttered by the user and turn them into a form that can be understood by the inference engine 11. The second function 9 is to take the output of the inference engine 11 and to send this back to the user's mobile phone 3.

In some embodiments, Natural Language Processing (NLP) is used in the interface 5. NLP helps computers interpret, understand, and then use everyday human language and language patterns. It breaks both speech and text down into shorter components and interprets these more manageable blocks to understand what each individual component means and how it contributes to the overall meaning, linking the occurrence of medical terms to the Knowledge Graph. Through NLP it is possible to transcribe consultations, summarise clinical records and chat with users in a more natural, human way.

However, simply understanding how users express their symptoms and risk factors is not enough to identify and provide reasons about the underlying set of diseases. For this, the inference engine 11 is used. The inference engine is a powerful set of machine learning systems, capable of reasoning on a space of >100s of billions of combinations of symptoms, diseases and risk factors, per second, to suggest possible underlying conditions. The inference engine can provide reasoning efficiently, at scale, to bring healthcare to millions.

In an embodiment, the Knowledge Graph 13 is a large structured medical knowledge base. It captures human knowledge on modern medicine encoded for machines. This is used to allows the above components to speak to each other. The Knowledge Graph keeps track of the meaning behind medical terminology across different medical systems and different languages.

In an embodiment, the patient data is stored using a so-called user graph 15.

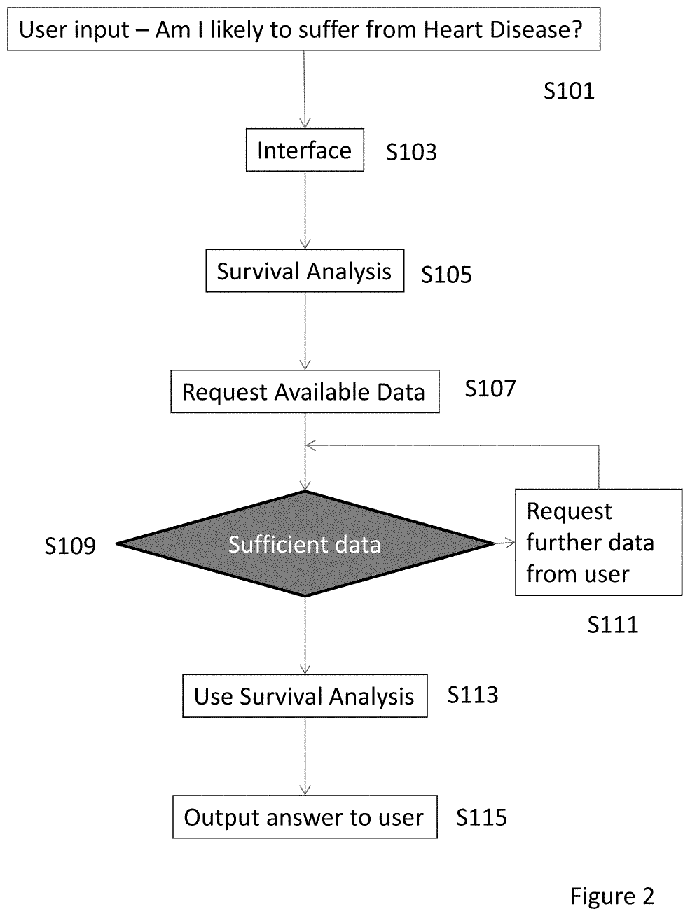

FIG. 2 is a flow diagram of a user submitting a query to the above system. In step S101, the user inputs a question using the system of FIG. 1, for example, “Am I likely to suffer from heart disease heart disease?”

This is then passed to the interface in step S103. The interface comprises various natural language processing algorithms that will allow the system to determine that the user is asking a question relating to the future health as opposed to a current diagnosis.

With this realised, the system passes to the survival analysis module in step S105. The system will request available data in step S107 that it has relating to the user. This can be data that is stored relating to the user. For example, if the user has previously used the system and stored their data. In a further embodiment, this can be data derived from measurements of the patient, for example, via a fitbit or the like.

In step S109, the system determines whether it has sufficient data. What is meant by sufficient data will differ dependent on the question asked by the user. For example, if the user wishes to understand their risk of heart disease the sufficient data required to determine this analysis may be different to that if the same user requested information concerning their chance of developing diabetes.

What is meant by sufficient data will be discussed in a little more detail later. However, the system will be able to answer the user's question with a certain confidence estimate. If the confidence estimate is too low, then the system will request further data from the user in step S111 which will allow the system to be able to determine the response with a higher confidence estimate.

The available data will comprise things such as the user's age, location and possibly past medical history. The survival analysis that will be then performed in step S113 uses the available data within an observable variable space. This is then used to construct a latent variable space which will be described later. The observable variable space will either use the values given by the user for the variables which it requires or it will use default value. Dependent on the question requested by the user, the system will require the user to input further values if the actual user data is required as opposed to the default value for certain questions.

Then in step S115, the answer is outputted to the user.

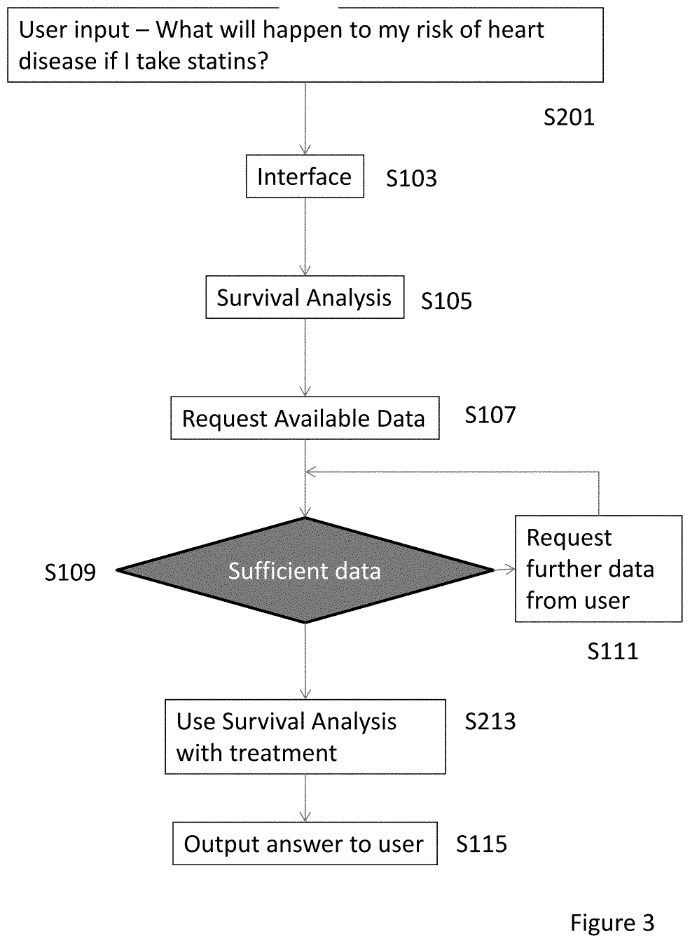

Before discussing the details of the model, FIG. 3 shows a different type of question that the model can also handle. Here, in step S201, the user inputs “what will happen to my risk of heart disease if I take statins?”. In this particular example, the user is asking the system to model the effect of an intervention or treatment.

To avoid unnecessary repetition, the same reference numerals will be used as in relation to FIG. 2 to denote the same features. Generally, the same process will be applied. However here, the survival analysis model that will be described below will indicate that a treatment/intervention (in this case the taking of Statins) will also need to be modelled.

Returning to the survival analysis step S113, the answer is predicted using a model. In an embodiment, first, a generative model is specified and it is assumed that the data is generated from this model.

In this embodiment, the following assumptions are made:

1) There is some latent space from which describes each individual, which takes the form of a multi-dimensional continuous variable Z. It is assumed that Z∈ [symbol] ^{D}^{_{z}}, where D_{z} is the dimensionality of the latent space.

2) There is a particular treatment variable of interest, T, with T∈{0, 1}. The value of T is determined by Z.
3) There is a set of proxy variables for the latent space, X. These can take the form of discrete covariates, X^{(disc)}∈{0, 1}, or of continuous covariates, X^{(cont)}∈ [symbol] . The value of X is determined by Z. The subscript j is used to denote each individual proxy variable, with j=1, . . . , D_{X}.
4) In this embodiment, there is a particular outcome of interest—the time-to-event variable, denoted by Y, with Y∈ [symbol] ⁺ The value of Y is determined by Z and T.

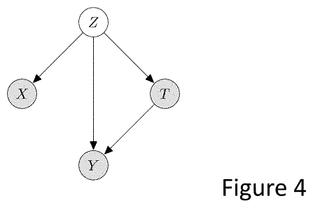

FIG. 4 denotes the causal links between these quantities.

The distributional assumptions of the model and the links between the variables will now be described.

The latent space Z defined in this embodiment, is drawn from a multivariate Normal distribution of zero mean and unit variance:

Z˜ [symbol] (0,1)

For the proxy variables, functions are defined to link the values of the latent space to the parameters for a Bernoulli distribution (for the discrete covariates) and a Normal distribution (for the continuous covariates):

Xⱼ^{(disc)}|Z˜Bernoulli(pⱼ), pⱼ=f₁(z) (1)

Xⱼ^{(cont)}|Z˜N(μⱼ,σⱼ²), μⱼ=f₂(z),σⱼ²=f₃(z) (2)

For the treatment variable, again a function is defined linking the latent space values to the parameter of a Bernoulli distribution:

a. T|Z˜Bernoulli(pₜ), pₜ=f₄(z) (3)

For the outcome, the distributional assumption depends on the chosen model architecture.

Variant 1 (Weibull):

In an embodiment, a Weibull distribution is used to explain Y. The scale parameter is determined by functions dependent on the latent space, selected conditional on the value of the treatment variable t. The shape parameter is chosen from fixed values k₀, k₁ dependent on t.

a. Y|T,Z˜Weibull(λ,kₜ), λ=(1−t)f₅(z)+tf₆(z) (4)

Variant 2 (PSSP):

Y is divided into a set of discrete, ordered labels denoting survival up to the time associated with the given discrete label. The probabilities of each label are described by a vector k=(k₀, k₁, . . . , k_{K}).

The “true” continuous time ŷ is mapped to a discrete label y_{τ} by the following function:

$$\begin{matrix}1 ) & \\ τ = [ \frac{\hat{y}}{{y}_{int}} ] - 1 , {y}_{int} = \frac{max ( Y )}{K} & ( 5 )\end{matrix}$$

max(Y) here denotes the “maximum” time-to-event value, determined either theoretically based on the problem or empirically from the available dataset. Values above this are placed in the final bucket y_{K} denoting that the event happens after the time associated with y_{K-1}.

The parameters k are determined by a function dependent on the latent space and selected conditional on the value of the treatment variable.

Y|T,Z˜Categorical(k), k=(1−t)f₅(z)+tf₆(z) (6)

The model framework expressed could be used in a number of capacities. Next, a specific example will be described relating to disease prediction and individualised treatment estimation.

In this example, the user inputs a query to understand their risk of heart attack, and whether the use of statins will help them specifically to reduce their risk.

In this context the variables are defined as follows:

Z: The latent space to be learned. This is unknown and estimated when the model is used to produce an answer, i.e. when in step S113 of FIG. 2 or step S213 of FIG. 3.

T: The treatment variable—e.g. take statins (t=1) or don't take statins t=0. In this example, both options would be explored for the user. Relating to FIGS. 1 and 2, t=0 corresponds to the question of FIG. 2, whereas t=1 corresponds to the question of FIG. 3.

X: The proxies for the latent confounding space. The exact nature of this will depend on the data available and relevant to the problem but in this example application could include synced device data from fitness trackers, previously recorded information for the individual, other available demographic information on the individual, and potentially additional information yielded via questionnaire. This data will be known and fixed at step S113 and S213.

Y: The time-to-event variable—the variable of prediction to learn when the user will develop a heart condition dependent on their current status and conditioned on their treatment options. This is unknown and predicted at step S113 or S213.

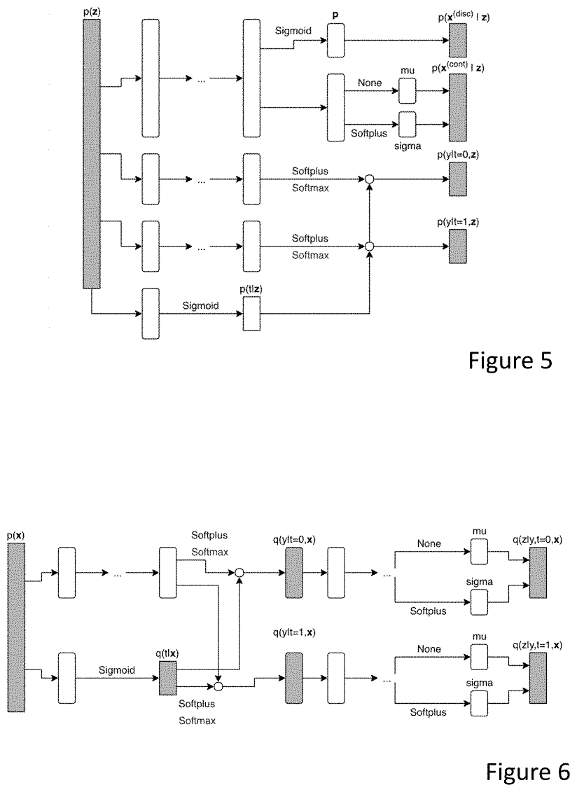

For the above problem a model is trained. The training will be described with reference to FIGS. 5 and 6.

In an embodiment, to train the model a longitudinal data set is used tracking the outcomes of a large number of individuals, available from existing longitudinal data sets (such as the UK Biobank) or other electronic health record storage.

In an embodiment, it is assumed that there is a dataset with N individuals indexed by i. The variable definitions for Xᵢ and Tᵢ remain the same (and indeed would be informed by the available data in the longitudinal studies).

Within the longitudinal data an event flag E is defined, which determines for a given individual's data whether the event of interest occurred or not during their time in the study. If Eᵢ=1, the event did occur, and the time-to-event variable Yᵢ is given by the observed value yᵢ*, yᵢ=yᵢ*. If Eᵢ=0, the event did not occur during the duration of the study for the user, and the time-to-event variable Yᵢ is known to be a value greater than or equal to the observed value yᵢ*, yᵢ≥yᵢ*. From the available data the latent space variable Zᵢ is estimated for each user from their observed data.

In an embodiment, Stochastic Variational Inference (SVI) is used to train the model. In an embodiment, this works by setting up a variational distribution q(zᵢ|xᵢ, tᵢ, yᵢ) to approximate the posterior probability of the latent space given the observed data, and using this to minimise the evidence lower bound (ELBO):

ELBO=Σᵢ₌₁^{N} [symbol] _{q}(zᵢ|xᵢ,tᵢ,yᵢ)[log p(xᵢ,tᵢ|zᵢ)+log p(zᵢ)+log p(yᵢ=yᵢ*|tᵢ,zᵢ,eᵢ=1)+log p(yᵢ≥yᵢ*|tᵢ,zᵢ;eᵢ=0)−log p(zᵢ|xᵢ,tᵢ,yᵢ)] (7)

Although SVI is mentioned above, other methods could be used, for example, Variational Inference (VI), Expectation Maximisation (EM), Expectation Propagation (EP). However, these methods may require more “bespoke” calculation for the training and/or approximations.

The ELBO term for the outcome variable yᵢ is dependent on the event flag eᵢ for the individual. The functions specifying the parameters for the distributions of the different variables in the models appearing in the ELBO from the model, f*(.), are specified by neural networks with parameter sets θ*. In practice these parameter sets overlap for different functions, as shared hidden layers are used for related variables. FIG. 5 gives an illustration of how these functions are parameterised. FIG. 5 shows an Illustration of the network architecture of the model. Distributions are marked by the shaded grey boxes. White boxes represent layers in the neural network architecture. The small circles indicate switching functionality to choose the relevant input based on the treatment t. Splits for different parameters and the activation functions where relevant are also included. Where two activation functions are written, the upper word denotes the function used in the Weibull variant whereas the lower text denotes the function to be used for PPSP.

The variational distribution (also referred to as guide) is defined as a multivariate Normal distribution with zero covariance between different dimensions of zᵢ:

zᵢ^{(guide)}|xᵢ,tᵢ,yᵢ˜ [symbol] (μᵢ,σᵢ²,I) (8)

μᵢ=(1−tᵢ)g₁(xᵢ,yᵢ,eᵢ)+(tᵢ)g₂(xᵢ,yᵢ,eᵢ) (9)

σᵢ²=(1−tᵢ)g₃(xᵢ,yᵢ,eᵢ)+(tᵢ)g₄(xᵢ,yᵢ,eᵢ) (10)

Here the functions g*(.) are also neural networks with parameter sets φ* There is again some level of shared representation between these functions—see FIG. 6 for more details That shows a network architecture of the guide. Distributions are marked by the shaded grey boxes and the same labelling is used as described above for FIG. 5.

The likelihood function requires additional terms for decoding where the choice of treatment variable and/or time to event variable is unknown. These decode estimates for these from xᵢ to get an accurate estimate for zᵢ prior to decoding the model proper.

In an embodiment, for treatment a probability q(tᵢ|xᵢ) can be specified where:

tᵢ^{(guide)}|xᵢ˜Bernoulli(pᵢ) pᵢ=g₅(xᵢ) (11)

For outcome a probability q(yᵢ|tᵢ, xᵢ).

Variant 1 (Weibull):

Here the shape parameter selection specified in the model is re-used. The scale parameter is set via a function on the proxy variables.

yᵢ^{(guide)}|tᵢ,xᵢ˜Weibull(λᵢ,kₜ_{ᵢ}), λᵢ=(1−tᵢ)g₆(xᵢ)+tᵢg₇(xᵢ) (12)

Variant 2 (PSSP):

Here the Categorical distribution formulation from the model is re-used.

yᵢ^{(guide)}|tᵢ,xᵢ˜Categorical(kᵢ), kᵢ=(1−tᵢ)g₆(xᵢ)+tᵢg₇(xᵢ) (13)

The final loss function is then specified as follows:

[symbol]

=ELBO=Σᵢ₌₁^{N}[log q(tᵢ=tᵢ*|xᵢ)+log q(yᵢ=yᵢ*|tᵢ,xᵢ;eᵢ=1)+log q(yᵢ≥yᵢ*|tᵢ,xᵢ;eᵢ=0)] (14)

The outputs of interest of the model are twofold: In an embodiment, the model is used to predict an individual's future outcome, p(Y|X,T), and in estimate the individual treatment effect (ITE) arising from intervening on the treatment variable (where do refers to do-calculus notation):

ITE(x)= [symbol] [Y|X=x,do(t=1)]− [symbol] [Y|X=x,do(t=0)] (15)

In further embodiments the model is used to estimate the population level treatment effect, known as the average treatment effect:

ATE= [symbol] [ITE(x)]

The above formulation of the problem using a latent confounder encoding allows the decoding of both objectives through the following approach:

1. Reconstruct the latent space zᵢ for an individual using the variational distribution functions.
2. Sample from the estimated latent space distribution and recover the downstream variables. Repeat for many samples to get an accurate estimation.
3. To recover prediction, decode the outcome variable using the existing setting of the treatment variable tᵢ.
4. To recover treatment effects, decode the outcome under the different conditions of t=0 and t=1 from the latent space estimated from the current (true) treatment variable setting.

Further details of the model and results can be found in Annex A.

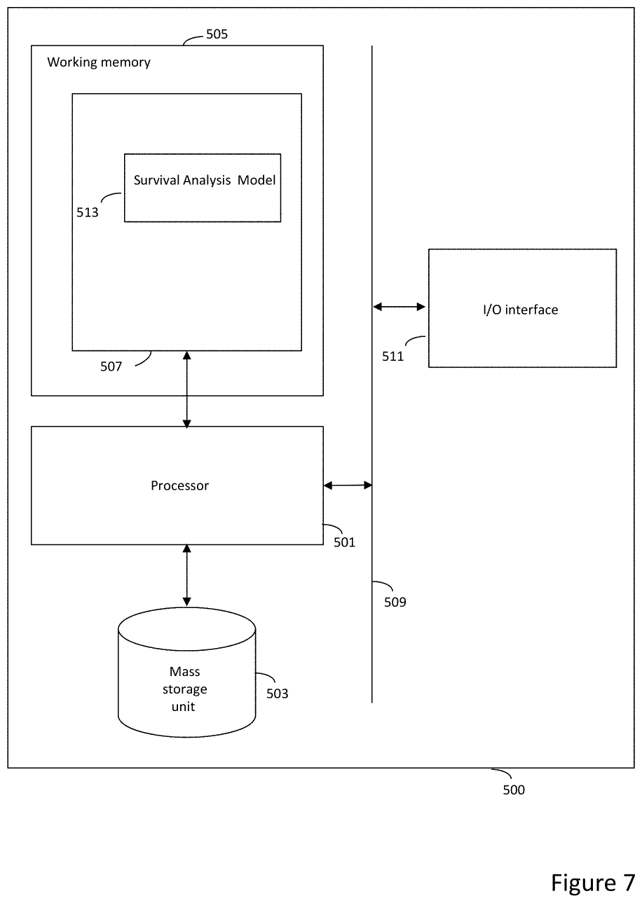

While it will be appreciated that the above embodiments are applicable to any computing system, an example computing system is illustrated in FIG. 7, which provides means capable of putting an embodiment, as described herein, into effect. As illustrated, the computing system 500 comprises a processor 501 coupled to a mass storage unit 503 and accessing a working memory 505. As illustrated, a survival analysis model 513 is represented as software products stored in working memory 505. However, it will be appreciated that elements of the survival analysis model 513, may, for convenience, be stored in the mass storage unit 503. Depending on the use, the survival analysis model 513 may be used with a chatbot, to provide a response to a user question that requires the survival analysis model.

Usual procedures for the loading of software into memory and the storage of data in the mass storage unit 503 apply. The processor 501 also accesses, via bus 509, an input/output interface 511 that is configured to receive data from and output data to an external system (e.g. an external network or a user input or output device). The input/output interface 511 may be a single component or may be divided into a separate input interface and a separate output interface.

Thus, execution of the survival analysis model 513 by the processor 501 will cause embodiments as described herein to be implemented.

The survival analysis model 513 can be embedded in original equipment, or can be provided, as a whole or in part, after manufacture. For instance, the survival analysis model 513 can be introduced, as a whole, as a computer program product, which may be in the form of a download, or to be introduced via a computer program storage medium, such as an optical disk. Alternatively, modifications to existing survival analysis model software can be made by an update, or plug-in, to provide features of the above described embodiment.

The computing system 500 may be an end-user system that receives inputs from a user (e.g. via a keyboard) and retrieves a response to a query using survival analysis model 513 adapted to produce the user query in a suitable form. Alternatively, the system may be a server that receives input over a network and determines a response. Either way, the use of the survival analysis model 513 may be used to determine appropriate responses to user queries, as discussed with regard to FIG. 1.

Implementations of the subject matter and the operations described in this specification can be realized in digital electronic circuitry, or in computer software, firmware, or hardware, including the structures disclosed in this specification and their structural equivalents, or in combinations of one or more of them. Implementations of the subject matter described in this specification can be realized using one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

While certain embodiments have been described, these embodiments have been presented by way of example only, and are not intended to limit the scope of the inventions. Indeed the novel methods and systems described herein may be embodied in a variety of other forms; furthermore, various omissions, substitutions and changes in the form of methods and systems described herein may be made without departing from the spirit of the inventions. The accompanying claims and their equivalents are intended to cover such forms of modifications as would fall within the scope and spirit of the inventions.

1. A computer implemented method of training a model, the model being used to predict whether a user will develop a health condition, the model being a probabilistic graphical model comprising a multidimensional observable variable space, a multidimensional latent variable space, an intervention variable space and a time to event variable, said time to event variable indicating when user is likely to develop a condition, wherein an observable variable space is dependent on a multidimensional latent space and the time to event variable is dependent on the multidimensional latent variable space and intervention variable space, wherein the intervention variable space models a treatment,
the model comprising a first statistical model comprising probability distributions linking the observable variable space to the multidimensional latent variable space and a second statistical model comprising probability distributions linking the time to event variable to the multidimensional latent variable space and intervention variable space,
the method comprising:
representing functional dependencies of the first and second statistical models as neural networks;
receiving training data comprising time to event data with corresponding intervention data and observable variables; and
training said neural networks using said training data.

2. The method of claim 1, wherein a probability of the time to event variable over the intervention variable space and the multidimensional latent variable space is an antisymmetric distribution.

3. The method of claim 2, wherein the probability of the time to event variable over the intervention variable space and the multidimensional latent variable space is a Weibull distribution.

4. The method of claim 1, wherein a probability of the time to event variable over the intervention variable space and the multidimensional latent variable space is a categorical distribution.

5. The method of claim 1, wherein the multidimensional latent variable space comprises both discrete and continuous variables.

6. The method of claim 1, wherein the multidimensional latent variable space is drawn from a multivariate Normal distribution.

7. The method of claim 1, wherein the multidimensional latent variable space comprises discrete variables and the observable variables are linked to the discrete variables of the multidimensional latent variable space via a Bernoulli probability distribution.

8. The method of claim 1, wherein the multidimensional latent variable space comprises continuous variables and the observable variables are linked to the continuous variables of the multidimensional latent variable space via a normal probability distribution.

9. A computer implemented method of determining whether a user will develop a health condition, the method comprising:
training a model, using observational training data wherein said observational training data comprises observations regarding individuals developing said condition, the model being a probabilistic graphical model comprising an observable variable space, a latent variable space and an outcome relating to said condition, wherein an observable multidimensional variable space is dependent on a multidimensional latent variable space and a likelihood of a user developing a condition is dependent on the multidimensional latent variable space;
retrieving data concerning the user,
inputting the retrieved data into said model; and
using said model to output if and when the user is likely to develop the condition.

10. The method of claim 9, wherein the model further comprises an intervention variable used to model intervention and wherein the likelihood of a user developing a condition is dependent on the latent variable space and the intervention variable.

11. The method of claim 9, wherein the likelihood of a user developing a condition is modelled as a time to event variable.
